(19) United States
(12) Patent Application Publication
(10) Pub. No.: US 2018/0280435 A1
(43) Pub. Date: Oct. 4, 2018

(54) T CELL DELIVERY OF MDA-7/IL-24 TO IMPROVE THERAPEUTIC ERADICATION OF CANCER AND GENERATE PROTECTIVE ANTITUMOR IMMUNITY

(71) Applicant: VIRGINIA COMMONWEALTH UNIVERSITY, Richmond, VA (US)

(72) Inventors: Paul B. FISHER, Henrico, VA (US); Xiang-Yang WANG, Glenn Allen, VA (US)

(21) Appl. No.: 15/766,212

(22) PCT Filed: Oct. 7, 2016
(86) PCT No.: PCT/US2016/055892

§ 371 Date: Apr. 5, 2018

Related U.S. Application Data
(60) Provisional application No. 62/239,579, filed on Oct. 9, 2015.

(51) Int. Cl. A61K 35/17; C12N 5/0783; C07K 14/54; A61K 45/06; A61P 35/00; A61P 35/04

(52) U.S. Cl. CPC A61K 35/17; C12N 5/0636; C07K 14/54; C12N 2510/00; A61P 35/00; A61P 35/04; C07K 14/5443; A61K 45/06

(57) ABSTRACT

Provided herein are methods and compositions useful for treating cancer, such as prostate cancer, through adoptive cell transfer of T cells derived from patients and genetically engineered to express MDA-7/IL-24 and/or other immune modulating agents. The methods described herein result in cancer cell death and reprogramming of the tumor immune compartment to restore antitumor immunity both at a primary tumor site and systemically.

### FIELD OF THE INVENTION

The invention generally relates to tumor-reactive or antigen-specific T lymphocytes derived from patients and genetically engineered to express MDA-7/IL-24 and/or other immune modulating agents for the treatment of cancer. The adoptive cell therapy results in eradication of both primary tumors and distant cancer metastases (e.g., lung, bone).

### BACKGROUND OF THE INVENTION

Despite significant advances in the understanding of the molecular and cellular changes involved in the initiation and progression of cancer, ninety percent of deaths can be attributed to metastatic disease, which are often resistant to conventional therapies (surgery, radiation and chemotherapy). This dismal scenario is principally due to the challenges posed by the complexity and heterogeneity of metastatic cancers, especially with regard to tumor dissemination and organ-specific colonization. As for most malignancies, mortality from prostate cancer (CaP), the most commonly diagnosed non-cutaneous cancer, derives from widespread metastases, including a propensity for bone. The standard first-line treatment of metastatic CaP is androgen deprivation therapy; however, over time virtually all patients progress to hormone-refractory disease.

Bone metastases are also associated with severe morbidity, pain and functional impairment. The absence of curative therapies for metastatic CaP emphasizes the crucial need to develop treatment strategies that are efficacious with minimal toxicity. No current single or combinatorial therapeutic approach has been effective in decreasing morbidity or engendering a cure for metastatic cancer within either soft tissue or bone. Consequently, developing novel targeted and effective therapies are compulsory to enhance survival.

Melanoma differentiation associated gene-7/Interleukin-24 (MDA-7/IL24), a secreted protein of the IL-10 family, functions as a cytokine at normal physiological levels and is expressed in tissues of the immune system. At supra-physiological levels, MDA-7/IL-24 plays a prominent role in inhibiting tumor growth, invasion, metastasis and angiogenesis and was recently shown to target tumor stem/initiating cells for death (see e.g., U.S. Pat. No. 7,579,330 incorporated herein by reference). MDA-7/IL-24 can selectively induce cell death in cancer cells without affecting normal cells. Thus, this gene originally shown to be associated with melanoma cell differentiation has now proven to be a multi-functional protein affecting a broad array of cancers. However, the majority of studies involve the use of adenoviruses to deliver MDA-7/IL-24, which may elicit a natural antiviral host response that subsequently limits antitumor efficacy of MDA-7/IL-24 and potentially provokes undesirable toxicity. Further, there is a risk that vector integration could cause malignant transformations of the host's tissues or cause unexpected complications related to the condition being treated. Thus, improved methods of delivering MDA-7/IL-24 to tumors are needed.

### SUMMARY OF THE INVENTION

Immunotherapy that harnesses the host immune system to fight cancer provides an important option for the treatment of cancer. T cells protect individuals from disease by targeting and eliminating diseased cells. Tumor-specific T cells can be isolated, followed by activation and expansion outside the body (in vitro), and then re-infused back into the patient to mediate cancer regression, a process termed adoptive T cell therapy.

One aspect of the invention provides a T lymphocyte genetically modified to express Melanoma differentiation associated gene-7/Interleukin-24 (MDA-7/IL-24) or a functional-conservative derivative thereof. In some embodiments, the MDA-7/IL-24 or functional- conservative derivative thereof comprises a FMS-like tyrosine kinase 3 (Flt-3) secretory motif. In some embodiments, the T lymphocyte is genetically modified to further express at least one of IL-15, IL-12, and MDA-5. In some embodiments, the method further comprises administering T lymphocytes, different from said T lymphocytes that express MDA-7/IL-24 or a functional-conservative derivative thereof, genetically modified to express at least one of IL-15, IL-12, and MDA-5.

Another aspect of the invention provides a composition for adoptive cell transfer comprising T lymphocytes as described herein and a pharmaceutically acceptable carrier. In some embodiments, the composition further comprises one or more chemotherapeutic or radiotherapeutic agents.

Another aspect of the invention provides a method of treating and preventing recurrence cancer in a subject in need thereof, comprising administering to the subject a therapeutically effective amount of a composition for adoptive cell transfer comprising T lymphocytes as described herein. In some embodiments, the T lymphocytes are isolated from said subject and genetically modified to express MDA-7/IL-24 or a functional-conservative derivative thereof. In some embodiments, the method further comprises a step of administering one or more of a chemotherapeutic or radiotherapeutic agent.

### BRIEF DESCRIPTION OF THE DRAWINGS

FIG. 1. Genetically-engineered T cells for eradication of CaP bone metastases. Tumor and/or antigen-specific T cell receptors direct activated T cells to locate and kill disseminated cancer cells, e.g. in the bone, based on antigen recognition and production of cytotoxic molecules, e.g., IFN-γ.

FIG. 2. IL-7/IL-15 expanded CD8+ T cells display a central memory phenotype. Antigen OVA-specific OT-I cells (upper) or lymphocytes from tumor-bearing mice (lower) were stimulated with Ionomycin (1 μM) and Bryostatin (5 nM) for 16 h. Cells were cultured in the presence of IL-7/IL-15 (10 ng/ml) for 6 d. For conventional T- cell expansion, cells were cultured with IL-2 (20 U/ml). FACS was performed by gating on CD8+ T cells.

FIG. 3. Antigen-specific T cells expanded by γ-chain cytokines display enhanced persistence in vivo. OT-I cells were stimulated with OVA257-264 (1 μg/mL) in the presence of IL-2 (40 IU/mL) or γ-chain cytokines (IL-7/IL-15, 10 ng/mL each) for 5 days. Cells (2×106 cells) were labeled with CFSE (5 μM) and transferred i.v. into C57BL/6 mice. Three days later, the frequency of CD8+CFSE+ T cells in lymph nodes (upper) and IFN-γ production by activated CD8+CFSE+ T cells (lower) was determined by FACS analysis and intracellular staining assays.

FIG. 4A-B. IL-15-engineered T cells are more effective than unmodified T cells in reducing lung metastases of CaP. A. RM1 tumor-sensitized T cells were engineered with a lentivirus expressing IL-15 following expansion. Production of IL-15 was assessed by ELISA. B. C57BL/6 mice were inoculated with 1×105 RM1 cells via tail vein to establish pulmonary metastases. 24 h later, mice received 107 T cells engineered to produce IL-15. Cells infected with viruses packaged using an empty vector serve as controls. p<0.01

FIG. 5A-B. IL-15 engineering enhances the trafficking of T cells to CaP bone metastatic niche. C57BL/6 mice were established with bone metastases by intracardiac injection of RMI-BM cells. 48 h later, mice received intravenously 107 IL-15-engineered, tumor-reactive T cells derived from CD45.1 transgenic mice. The presence of transferred T cells in bone marrow cavity of hind limbs were analyzed for CD45.1+CD8+ cells using FACS (A). The expression of PD-1 expression on these cells were also examined (B).

FIG. 6. Potential immune modulation by MCL-1 inhibition. MCL-1 inhibitor reduces MDSCs in tumor-bearing mice. Tumor-bearing mice received BI-97C1 (Sabutoclax) (5 mg/kg or 20 mg/kg) i.p. or left untreated. 24 h later, splenic CD11b+GR-1+MDSCs were assessed by FACS.

FIG. 7A-C. Ad-mediated MDA-7/IL-24 expression can eradicate primary and inhibit distant CaP xenografts in nude mice and restrain tumor development in Hi-Myc transgenic mice. A. Therapy resistant prostate tumors (PC-3 cells stably overexpressing Bcl-2) were established in both right and left flanks; and only tumors on the left side were injected either with tropism-modified cancer terminator virus (Ad.5/3-CTV),77 that permits replication uniquely in cancer cells with simultaneous production of MDA-7/IL-24, or controls (Vector control: Ad.5-vec, Ad.⁵/₃-vec; Replicating virus with MDA-7/IL-24: Ad.5-PEG-E1A, Ad.5/3-PEG-E1A; Non tropism modified cancer terminator virus Ad.5-CTV). Representative photographs of the prostate tumors at the end of the study are shown. B. BLI was performed using Xenogen In Vivo Imaging System (IVIS). C. Therapeutic virus Ad.5/3-CTV was delivered in the prostatic region of 22 week-old male Hi-Myc mice, a transgenic model for spontaneous prostate carcinoma, through UTMD (ultrasound-targeted microbubble-destruction) approach. The paraffin-embedded prostate sections were immunostained for MDA-7/IL-24. TUNEL assay and KI-67 staining were used to detect cell apoptosis and proliferation.

FIG. 8A-B. MDA-7/IL-24 displays immunomodulatory activity. A. CD8+ T cells were stimulated with anti-CD3/CD28 antibodies in the presence or absence of purified MDA-7/IL-24 protein (20 ng/ml). Cell proliferation was measured using 3H-thymidine (TdR) incorporation assays. P<0.05. B. C57BL/6 mice were injected i.v. with MDA-7/IL-24 protein (10 μg) or PBS. 48 h later, lymph node cells were assessed for IFN-γ-producing CD8+ T cells by intracellular staining.

FIG. 9A-C. (A) Schematic diagram of MDA-7 and M4. Structure prediction model using SWISS-MODEL of (B) MDA-7 and (C) M4.

FIG. 10A-C. MDA-7 modified T cells exhibit antigen-dependent and independent cytotoxicity. (A) Freshly isolated RM1-OVA tumor sensitized T cells or IL-7/IL-15-expanded T cells were co-cultured with OVA peptide loaded BMDC, followed by ELISPOT analysis for OVA-reactive, IFN-γ-producing T cells. (B) IL-7/IL-15 expanded OT-I T cells were transduced with LV-Vector, LV-MDA-7 or left unmodified, and then co-cultured with RM1-OVA or parental RM1 tumor cells at 20:1 ratio for 24 h. Cytotoxicity was determined using LDH assay. (C) RM1-OVA tumor- sensitized T cells were engineered with LV-MDA-7 or LV-Vec, and subjected to cytolytic assays against RM1-OVA tumor cells. **p<0.01.

FIG. 11A-D. MDA-7 engineering enhances the therapeutic potency of adoptive T cell therapy against metastatic CaP. C57BL/6 mice were established with experimental lung metastases by i.v. injection of 1×10⁵ RM1-Luc tumor cells. Six days later, mice were treated with or without RM1 tumor sensitized T cells (10⁷ cells) that were modified with either vector or MDA-7. Lung tumor metastases were monitored by bioluminescent imaging (A). Lungs were collected from treated mice and prepared as single cell suspensions, followed by clonogenic formation assays (B). C57BL/6 mice established with lung metastases of RM1 tumors were treated as described. Lung infiltration of CD90.1+CD8 or CD4 T cells was analyzed 72 hours after adoptive transfer (C). The transcriptional activation of genes Ifng and Il12p35 in the lungs with metastases was examined using RT-PCR analyses. **, p<0.01

FIG. 12A-C. MDA-7 modification improves therapeutic activity of T cell therapy in the treatment of pre-established subcutaneous tumors. C57BL/6 mice were inoculated s.c. with 2×10⁵ B16 tumor cells. On day 5, mice received tumor-sensitized T cells engineered to produce MDA-7. Mice without treated or treated with vector modified T cells (10⁷ cells) served as controls. Tumor growth was monitored every other days (A). Tumor infiltrating CD4⁺or CD8⁺ T cell subsets as well as adoptively transferred CD90.1⁺ CD8⁺ T cells were examined by FACS (B). The ratio of tumor-associated T effector cells (CD3⁺ CD8⁺) or T helper cells (CD4⁺FoxP3⁻) vs T regulatory cells (CD4⁺FoxP3⁻) is also presented (C). ** p<0.01.

### DETAILED DESCRIPTION OF THE INVENTION

The invention generally provides applications of Tcell-based immunotherapy and strategies to overcome tumor-induced immune suppression for eradicating cancer, in particular, prostate cancer (CaP) and CaP bone metastases. Immunotherapy based on the adoptive transfer of naturally occurring or genetically engineered T lymphocytes mediates tumor regression in patients with metastatic cancer by recognizing tumor antigens present on their surface. The stimulation of immune responses by adoptive cell therapy (ACT) can usually be accomplished without causing toxicity, mostly due to the high sensitivity and specificity of tumor-reactive T cells.

Adoptive T cell therapy has been used previously in cancer therapy, e.g. to express chimeric antigen receptor (CAR) for recognition of specific antigens (see US20140004132 herein incorporated by reference). The strategy provided herein differs significantly from these approaches in that it is an object of the invention to use T cells to specifically deliver unique antitumor agents (e.g., MDA-711L-24) to tumor sites, which engages additional, non-overlapping antitumor pathways in addition to T cell-mediated tumor killing for synergistic anticancer efficacy or to further strengthen the effector functions of T cells (e.g., IL-12, IL-15, MDA-5) by reprograming the tumor environment (e.g. promote IFN-beta production).

This safe and efficient platform, which is based on unique tumor-recognition capacity of T lymphocytes, will increase the specificity of tumor targeted-delivery of MDA-7/IL-24 or other therapeutic agents, while also noticeably improving its antitumor efficacy through the use of high-avidity T lymphocytes. Specific delivery of therapeutic agents to the tumor sites will also reduce side effects compared to systemic administration. An exemplary MDA-7/IL-24 DNA and amino acid sequence is represented by SEQ ID NO: 1 and SEQ ID NO: 2, respectively (see also GenBank Accession No. NM_006850.3). An active part or all of the entire sequence may be incorporated into the cells of the invention.

The clinical application of ACT has been limited by the escape mechanisms of tumor cells that avoid immune destruction due to immunoediting (e.g., selection of non-immunogenic or antigen loss cancer cell variants). Since cancer-specific toxicity of MDA-7/IL-24 is antigen-independent, the delivery of this cancer-suicide agent by T cells overcomes the acquired tumor resistance to ACT and additionally enables ACT to achieve its optimum therapeutic potential. The combination of multivalent tumor-specific killing (i.e., immune-mediated and non-immune-mediated tumor destruction, and tumor toxicity) in a single personalized treatment regimen offers unique advantages for clinical applications. The superiority of tumor-specific T cells to seek and eradicate cancer cells combined with the selective tumoricidal as well as potential immune-modulating activity of MDA-7/IL-24 generates long-lasting immune protection. Thus, the methods described herein assist not only in managing primary tumors and normally inaccessible metastatic disease, but additionally prevent relapse when used in conjunction with other treatment modalities.

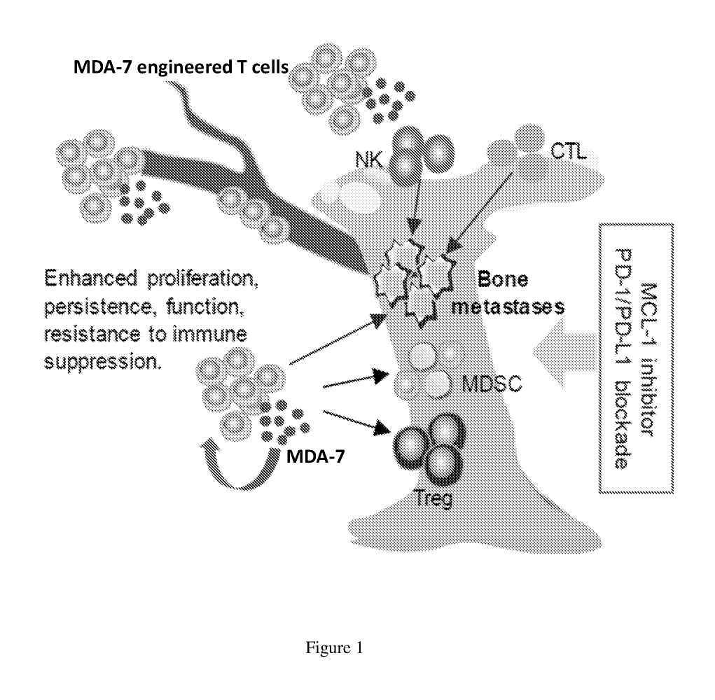

Elimination of CaP bone lesions using the methods as described herein, i.e. through a T-cell-mediated immune mechanism, establishes antitumor immunity, which prevents cancer recurrence. In addition to those diagnosed with metastatic CaP, this adoptive T-cell therapy also provides prophylactic activity to protect patients at high-risk for metastatic CaP from developing bone metastases (FIG. 1). As shown in FIG. 1, engineering T cells with MDA-7/IL-24, for example, promotes the survival, expansion, and function of T cells. MDA-7/IL-24 also renders T cells resistant to the immunosuppression by Tregs and MDSCs in the tumor microenvironment (TME). Targeted delivery of MDA-7/IL-24 to the metastatic sites can reprogram and modify the TME, resulting in mobilization of endogenous innate and adaptive immune cells for coordinated elimination of bone metastases and consequently providing protective immunity against relapse. The approaches to counteract the immunosuppressive pathways in the TME (e.g., MCL-1 inhibitor, immune checkpoint blockade) improve the potency of T-cell therapy to eradicate metastatic CaP.

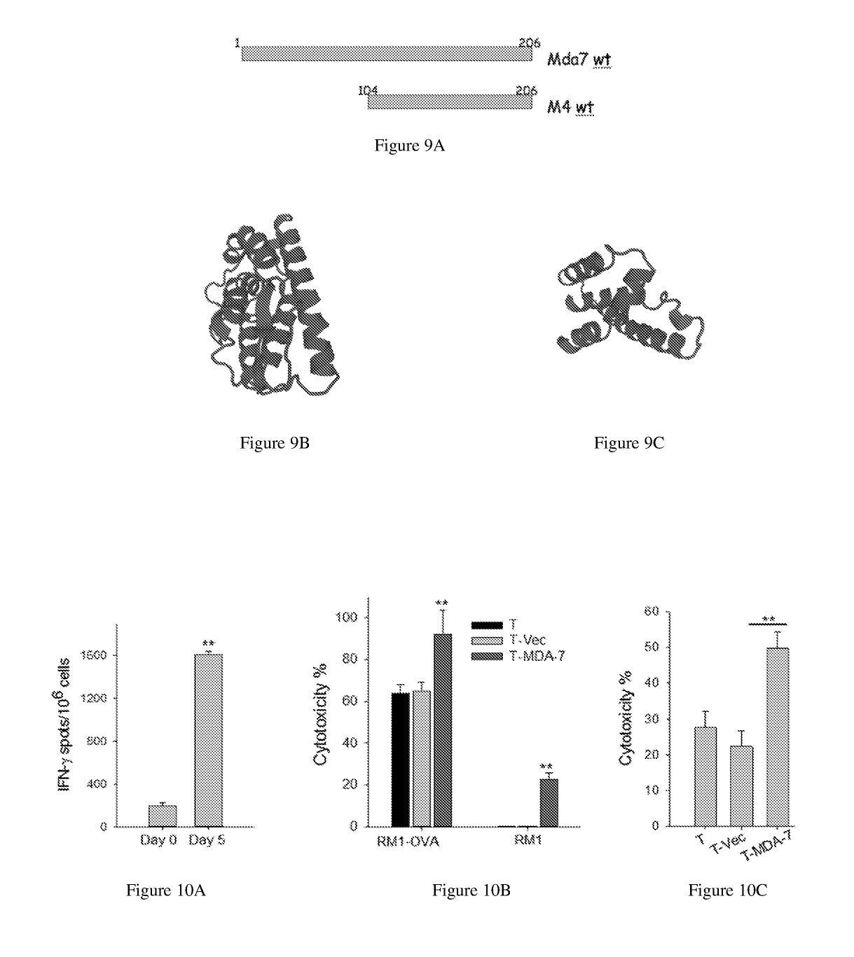

A functional-conservative derivative of MDA-7 includes, but it not limited to, a “therakine”, which comprises a portion, or active component, of MDA-7with a Flt-3 secretory motif, that has been shown to display properties similar to MDA-7. The portion of MDA-7 present in the therakine is termed M4 with the DNA and amino acid sequences represented by SEQ ID NO: 3 and SEQ ID NO: 4, respectively. A schematic diagram and structure prediction models of MDA-7 and M4 are shown in FIG. 9A-C.

As provided herein, the use of tumor-reactive or antigen-specific T cells as a shuttle vehicle circumvents the issues associated with viral delivery and, more importantly, ensure specific and efficient targeting of therapeutic MDA7/IL-24 of functional derivatives thereof to tumors, including metastatic sites. Consequently, delivering a cancer-suicide agent by T cells that embodies their intrinsic tumor-killing capability enables adoptive T cell therapy to reach its maximum therapeutic potential. Furthermore, these tumor-reactive T cells can be used to deliver other therapeutic or immune modulating agents, alone or in combination, including but not limited to MDA-7/IL-24, “therakine”, IL-15, IL-12, and MDA-5. In some embodiments, the method further comprises administering T lymphocytes, different from said T lymphocytes that express MDA-7/IL-24 or a functional-conservative derivative thereof, genetically modified to express at least one of IL-15, IL-12, and MDA-5.

Concurrent engagement of multivalent antitumor mechanisms via the T cell vehicle that specifically recognize cancer cells achieves optimal control of cancers and metastases.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness.

The term “cancer metastasis” has its general meaning in the art and refers to the spread of a tumor from one organ or part to another non-adjacent organ or part.

Any cancer or metastatic cancer may be targeted using the inventive therapy including, but not limited to, prostate, brain, breast, pancreatic, liver, kidney, lung, spleen, gall bladder, anal, testicular, ovarian, cervical, skin, bone, blood, and colon. In some embodiments, the T-lymphocytes described herein are used to treat bone metastasis of prostate cancer. The methods described herein can eradicate bone metastases and prevent relapse. Arming T cells with MDA-7/IL-24 enhances the magnitude and durability of the antitumor immune response, resulting in long-term protective immunity against relapse.

The terms “subject” and “patient” are used interchangeably herein, and refer to an animal such as a mammal, which is afflicted with or suspected of having, at risk of, or being pre-disposed to cancer. The terms may refer to a human. The terms also include domestic animals bred for food, sport, or as pets, including horses, cows, sheep, poultry, fish, pigs, cats, dogs, and zoo animals, goats, apes (e.g. gorilla or chimpanzee), and rodents such as rats and mice. Typical subjects include persons susceptible to, suffering from or that have suffered from cancer.

T lymphocytes can be isolated from tumor or peripheral blood of the individual to be treated by methods known in the art and cultured in vitro. Lymphocytes are cultured in media such as RPMI or RPMI 1640 for about 4 days to 2 weeks. Viability is assessed by trypan blue dye exclusion assay. Cytokines may be added to the lymphocyte culture such as IL-2 or IL-15.

The T lymohocytes of the invention are ex vivo cultured cells that recombinantly express an anti-cancer agent as described herein. In some embodiments, the T lymphocytes are tumor-reactive or antigen-specific T lymphocytes. The T lymphocytes may comprise a polynucleotide (such as an expression vector) that encodes an active part or all of MDA-7/IL-24. The cells may be transformed or transfected with such a vector. A vector in the cells may comprise an expression construct that encodes MDA-7/IL-24, “therakine”, IL-15, IL-12, MDA-5, or a combination thereof. A single vector may comprise an expression construct that encodes the anti-cancer agents described herein, or multiple vectors may comprise expression constructs that encode the anti-cancer agents. In cases where an expression construct encodes two or more of the anti-cancer agents, their regulation of expression may be directed by the same or by different regulatory elements. In certain embodiments, the two or more of the anti-cancer agents are expressed as a single polycistronic polypeptide in which the individual polypeptides are separated by a cleavable peptide; e.g., 2A peptide. Illustrative examples of expression vectors include, but are not limited to, a plasmid or viral vector.

As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells that do not contain a recombinantly introduced nucleic acid.

The MDA-7/IL-24 constructs can be introduced as one or more DNA molecules or constructs, where there may be at least one marker that will allow for selection of host cells that contain the construct(s). The constructs can be prepared in conventional ways, where the genes and regulatory regions may be isolated, as appropriate, ligated, cloned in an appropriate cloning host, analyzed by restriction or sequencing, or other convenient means. Particularly, using PCR, individual fragments including all or portions of a functional unit may be isolated, where one or more mutations may be introduced using “primer repair”, ligation, in vitro mutagensis, etc. as appropriate. The construct(s) once completed and demonstrated to have the appropriate sequences may then be introduced into the host cell by any convenient means. The constructs may be integrated and packaged into non-replicating, defective viral genomes like lentivirus, Adenovirus, Adeno-associated virus (AAV), or Herpes simplex virus (HSV) or others, including retroviral vectors, for infection or transduction into cells. The constructs may include viral sequences for transfection, if desired. Alternatively, the construct may be introduced by fusion, electroporation, biolistics, transfection, lipofection, or the like. The host cells may be grown and expanded in culture before introduction of the construct(s), followed by the appropriate treatment for introduction of the construct(s) and integration of the construct(s). The cells are then expanded and screened by virtue of a marker present in the construct. Various markers that may be used successfully include hprt, neomycin resistance, thymidine kinase, hygromycin resistance, etc.

In specific embodiments, MDA-7/IL-24 is introduced into the cells as an RNA for transient expression. RNA can be delivered to the immune cells of the disclosure by various means including microinjection, electroporation, and lipid-mediated transfection, for example. In particular aspects, introduction of constructs into cells may occur via transposons. An example of a synthetic transposon for use is the Sleeping Beauty transposon that comprises an expression cassette including the heparanase gene of active fragment thereof.

In some instances, one may have a target site for homologous recombination, where it is desired that a construct be integrated at a particular locus. For example, one can knock-out an endogenous gene and replace it (at the same locus or elsewhere) with the gene encoded for by the construct using materials and methods as are known in the art for homologous recombination. For homologous recombination, one may use either .OMEGA, or O-vectors. See, for example, Thomas and Capecchi, 1987; Mansour, et ah, 1988; and Joyner, et al, 1989.

The constructs may be introduced as a single DNA molecule encoding at least MDA-7/IL-24 and optionally another gene, or different DNA molecules having one or more genes. The constructs may be introduced simultaneously or consecutively, each with the same or different markers. In an illustrative example, one construct would contain MDA-7/IL-24 under the control of particular regulatory sequences.

Vectors containing useful elements such as bacterial or yeast origins of replication, selectable and/or amplifiable markers, promoter/enhancer elements for expression in prokaryotes or eukaryotes, etc. that may be used to prepare stocks of construct DNAs and for carrying out transfections are well known in the art, and many are commercially available.

The cells that have been modified to express MDA-7/IL-24 (such as with DNA constructs) may be grown in culture under selective conditions, and cells that are selected as having the construct may then be expanded and further analyzed, using, for example; the polymerase chain reaction for determining the presence of the construct in the host cells. Once the modified host cells have been identified, they may then be used as planned, e.g. expanded in culture or introduced into a host organism.

Depending upon the nature of the cells, the cells may be introduced into a host organism, e.g. a mammal, in a wide variety of ways. The cells are introduced at the site of the tumor, in specific embodiments, although in alternative embodiments the cells hone to the cancer or are modified to hone to the cancer. The number of cells that are employed will depend upon a number of circumstances, the purpose for the introduction, the lifetime of the cells, the protocol to be used, for example, the number of administrations, the ability of the cells to multiply, the stability of the recombinant construct, and the like. The cells may be applied as a dispersion, generally being injected at or near the site of interest. The cells may be in a physiologically-acceptable medium.

In particular embodiments, the route of administration may be intravenous, intraarterial, intraperitoneal, or subcutaneous, for example. Multiple administrations may be by the same route or by different routes.

Functional-conservative derivatives or variants may result from modifications and changes that may be made in the structure of a polypeptide (and in the DNA sequence encoding it), and still obtain a functional molecule with desirable characteristics (e.g. tumoricidal and/or immunostimulatory effects). Functional-conservative derivatives may also consist of a fragment of a polypeptide that retains its functionality (e.g., the “therakine” as described herein).

Accordingly, functional-conservative derivatives or variants are those in which a given amino acid residue in a protein has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A functional-conservative derivative also includes a polypeptide which has at least 60% amino acid identity as determined by BLAST or FASTA algorithms, preferably at least 75%, more preferably at least 85%, still preferably at least 90%, and even more preferably at least 95%, and which has the same or substantially similar properties or functions as the native or parent protein to which it is compared. Two amino acid sequences are “substantially homologous” or “substantially similar” when greater than 80%, preferably greater than 85%, preferably greater than 90% of the amino acids are identical, or greater than about 90%, preferably greater than 95%, are similar (functionally identical). Preferably, the similar or homologous sequences are identified by alignment using, for example, the GCG (Genetics Computer Group, Program Manual for the GCG Package, Version 7, Madison, Wisconsin) pileup program, or any of sequence comparison algorithms such as BLAST, FASTA, etc.

For example, certain amino acids may be substituted for other amino acids in a protein structure without appreciable loss of tumoricidal effects. Since it is the interactive capacity and nature of a protein that defines that protein's biological functional activity, certain amino acid substitutions can be made in a protein sequence, and, of course, in its DNA encoding sequence, and nevertheless obtain a protein with like properties. It is thus contemplated that various changes may be made in the polypeptide sequences of the invention, or corresponding DNA sequences which encode said polypeptides, without appreciable loss of their biological activity. Said tumoricidal activity and immunostimuolatory activity can be assessed by various techniques well-known in the art, such as for instance the assays referred in the Example.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine.

The term “treating” or “treatment”, as used herein, means reversing, alleviating, inhibiting the progress of, or ameliorating the disorder or condition to which such term applies, or one or more symptoms of such disorder or condition. For example, the treatment of the invention may slow the growth of said cancer, reduce the number of tumor cells in said cancer, reduce tumor load, or eliminate said cancer.

The term “prevent” or “prevention” refers to any success or indicia of success in the forestalling or delay of cancer recurrence/relapse in patients in clinical remission, as measured by any objective or subjective parameter, including the results of a radiological or physical examination. The patient may have cancer at the time of treatment (and thus future recurrence of the cancer is prevented) or may be in remission, e.g. after treatment with the agents of the invention and/or another course of therapy. Thus, the agents of the invention may be used as an anti-cancer vaccine.

By a “therapeutically effective amount” is meant a sufficient amount of the molecule to treat a cancer, (for example, to limit tumor growth or to slow or block tumor metastasis) at a reasonable benefit/risk ratio applicable to any medical treatment. It will be understood, however, that the total daily usage of the molecules and compositions of the present invention will be decided by the attending physician within the scope of sound medical judgment. The specific therapeutically effective dose level for any particular subject will depend upon a variety of factors including the disorder being treated and the severity of the disorder; activity of the specific polypeptide employed; the specific composition employed, the age, body weight, general health, sex and diet of the subject; the time of administration, route of administration, and rate of excretion of the specific polypeptide employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved.

The T cell therapy described herein may be combined with other strategies, including a US Food and Drug Administration approved immune modulator (i.e., pembrolizumab) to overcome the immunosuppressive pathways in bone metastatic niche for amplification of antitumor immune functions. Besides providing therapeutic benefit to cancer patients with established metastatic disease, this approach may also be used in an adjuvant setting in combination with other standard-of-care treatments (e.g., radiation therapy, hormonal therapy) to protect patients at high risk of metastatic disease from developing bone metastases.

Immune checkpoint inhibitors (e.g., anti-PD-1/PD-L1 antibodies such as pembrolizumab) as well as an Mcl-1 inhibitor (Sabutoclax), an apoptosis-based cancer therapeutic that can also target osteoclast differentiation and the immune niche, may also be used to reprogram the microenvironment of cancer metastases to improve and optimize adoptive T-cell therapy, e.g. by preventing inactivation of T cells. Immune checkpoint inhibitors and/or Mcl-1 inhibitors may be administered sequentially or concomitantly with the T lymphocytes of the invention.

In a particular embodiment the T lymphocytes of the invention may be administered sequentially or concomitantly with one or more chemotherapeutic or radiotherapeutic agents.

In one embodiment said chemotherapeutic or radiotherapeutic agents are a therapeutic active agent used as anticancer agent. For example, said anticancer agents include but are not limited to fludarabine, gemcitabine, capecitabine, methotrexate, mercaptopurine, thioguanine, hydroxyurea, cytarabine, cyclophosphamide, ifosfamide, nitrosoureas, platinum complexes such as cisplatin, carboplatin and oxaliplatin, mitomycin, dacarbazine, procarbazine, epipodophyllotoxins such as etoposide and teniposide, camptothecins such as irinotecan and topotecan, bleomycin, doxorubicin, idarubicin, daunorubicin, dactinomycin, plicamycin, mitoxantrone, L-asparaginase, doxorubicin, epirubicin, 5-fluorouracil and 5-fluorouracil combined with leucovorin, taxanes such as docetaxel and paclitaxel, levamisole, estramustine, nitrogen mustards, nitrosoureas such as carmustine and lomustine, vinca alkaloids such as vinblastine, vincristine, vindesine and vinorelbine, imatinib mesylate, hexamethylmelamine, kinase inhibitors, phosphatase inhibitors, ATPase inhibitors, tyrphostins, protease inhibitors, inhibitors herbimycin A, genistein, erbstatin, and lavendustin A. In one embodiment, additional anticancer agents may be selected from, but are not limited to, one or a combination of the following class of agents: alkylating agents, plant alkaloids, DNA topoisomerase inhibitors, anti-folates, pyrimidine analogs, purine analogs, DNA antimetabolites, taxanes, podophyllotoxins, hormonal therapies, retinoids, photosensitizers or photodynamic therapies, angiogenesis inhibitors, antimitotic agents, isoprenylation inhibitors, cell cycle inhibitors, actinomycin, bleomycin, anthracyclines, MDR inhibitors and Ca²⁺ ATPase inhibitors.

Additional anticancer agents may be selected from, but are not limited to, cytokines, chemokines, growth factors, growth inhibitory factors, hormones, soluble receptors, decoy receptors, monoclonal or polyclonal antibodies, mono-specific, bi-specific or multi-specific antibodies, monobodies, polybodies.

Further therapeutic active agents may be an antiemetic agent. Suitable antiemetic agents include, but are not limited to, metoclopramide, domperidone, prochlorperazine, promethazine, chlorpromazine, trimethobenzamide, ondansetron, granisetron, hydroxyzine, acetylleucine, alizapride, azasetron, benzquinamide, bietanautine, bromopride, buclizine, clebopride, cyclizine, dimenhydrinate, diphenidol, dolasetron, meclizine, methallatal, metopimazine, nabilone, pipamazine, scopolamine, sulpiride, tetrahydrocannabinols, thiethylperazine, thioproperazine and tropisetron. In a preferred embodiment, the antiemetic agent is granisetron or ondansetron.

In still another embodiment, the other therapeutic active agent can be an opioid or non-opioid analgesic agent. Suitable opioid analgesic agents include, but are not limited to, morphine, heroin, hydromorphone, hydrocodone, oxymorphone, oxycodone, metopon, apomorphine, buprenorphine, meperidine, loperamide, ethoheptazine, betaprodine, diphenoxylate, fentanyl, sufentanil, alfentanil, remifentanil, levorphanol, dextromethorphan, phenazone, pemazocine, cyclazocine, methadone, isomethadone and propoxyphene. Suitable non-opioid analgesic agents include, but are not limited to, aspirin, celecoxib, rofecoxib, diclofenac, diflunisal, etodolac, fenoprofen, flurbiprofen, ibuprofen, ketoprofen, indomethacin, ketorolac, meclofenamate, mefenamic acid, nabumetone, naproxen, piroxicam and sulindac.

In yet another embodiment, the further therapeutic active agent can be an anxiolytic agent. Suitable anxiolytic agents include, but are not limited to, buspirone, and benzodiazepines such as diazepam, lorazepam, oxazapam, clorazepate, clonazepam, chlordiazepoxide and alprazolam.

The term “radiotherapeutic agent” as used herein, is intended to refer to any radiotherapeutic agent known to one of skill in the art to be effective to treat or ameliorate cancer, without limitation. For instance, the radiotherapeutic agent can be an agent such as those administered in brachytherapy or radionuclide therapy. Such methods can optionally further comprise the administration of one or more additional cancer therapies, such as, but not limited to, chemotherapies, and/or another radiotherapy.

Another aspect of the invention relates to a pharmaceutical composition comprising a T lymphocyte according to the invention and a pharmaceutically acceptable carrier. Pharmaceutically” or “pharmaceutically acceptable” refer to molecular entities and compositions that do not produce an adverse, allergic or other untoward reaction when administered to a subject, such as a human, as appropriate. A pharmaceutically acceptable carrier or excipient refers to a non-toxic solid, semi-solid or liquid filler, diluent, encapsulating material or formulation auxiliary of any type.

The T lymphocytes of the invention may be contained in physiological saline, phosphate buffered saline (PBS), culture medium, or the like in order to maintain stability.

In the pharmaceutical compositions of the present invention for oral, sublingual, subcutaneous, intramuscular, intravenous, intratumoral, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the T lymphocytes in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

In addition to the compositions of the invention formulated for parenteral administration, such as intravenous or intramuscular injection, other pharmaceutically acceptable forms include, e.g. tablets or other solids for oral administration; liposomal formulations; time release capsules; and any other form currently used.

Any of the compositions described herein may be comprised in a kit. In a non-limiting example, one or more cells for use in cell therapy that harbors recombinantly expressed heparanase and/or the reagents to generate one or more cells for use in cell therapy that harbors recombinantly expressed heparanase may be comprised in a kit. The kit components are provided in suitable container means. In specific embodiments, the kits comprise recombinant engineering reagents, such as vectors, primers, enzymes (restriction enzymes, ligase, polymerases, etc.), buffers, nucleotides, etc.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the components in close confinement for commercial sale. Such containers may include injection or blow-molded plastic containers into which the desired vials are retained.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

### EXAMPLE 1

### Applications of T-Cell-Based Immunotherapy and Strategies to Overcome Tumor-Induced Immune Suppression for Eradicating CaP Bone Metastases

### Introduction

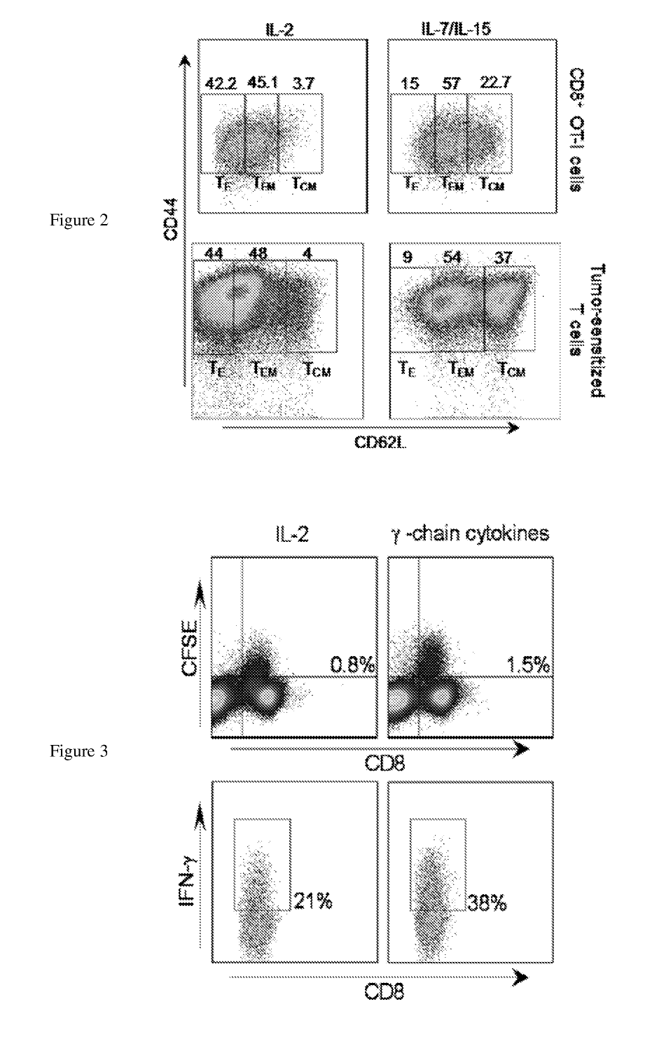

The long-term antitumor potential of T lymphocytes depends on the ability of the cells to persist, self-renew, and differentiate into antitumor effectors¹ and thus, on the degree of differentiation of such T cells.² The cytokine IL-15 is known to drive a durable immune response by promoting memory T cells,³⁻⁶ which have been shown to be superior in conferring long-term protective immunity against infectious disease and cancer.⁷⁻¹¹ Using an ex vivo protocol involving Bryostatin/ Ionomycin (B/I) and common γ-chain cytokines (IL-7/IL-15),¹² we efficiently and preferentially expanded antigen-specific or tumor-reactive CD8+ T cells with a central memory phenotype (CD44+CD62L^{high}, FIG. 2) that supports enhanced in vivo antitumor efficacy.^{13,14} In particular, these programed CD8+ T cells displayed significantly prolonged survival and/or persistence in vivo compared to those expanded by IL-2 (FIG. 3).

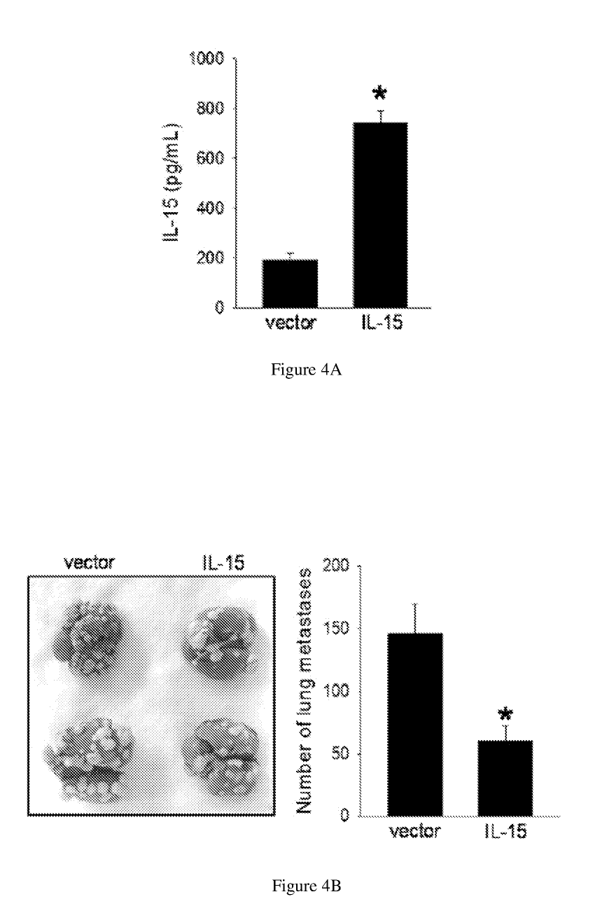

Our studies and others also showed that IL-15 can render T cells resistant to immunosuppressive Tregs, or myeloid-derived suppressive cells (MDSCs),¹⁵ which are part of an array of immunosuppressive components present in the TME. These results underscore a critical role of γ-chain cytokines or IL-15 in homeostatic proliferation and survival of mature lymphocytes,^{16,17} and provide a rationale for using IL-15-engineered T cells to eradicate CaP bone metastases. Indeed, IL-15 is now at the top of the NCI's list of agents with the greatest potential use in cancer immunotherapy.¹⁸ Our study showed that T cells genetically modified to express IL-15 were more effective than unmodified cells in suppressing lung metastases of mouse CaP (FIG. 4A-B).

These results demonstrate that tumor/antigen-reactive T cells engineered to produce IL-15 are highly effective in destroying disseminated CaP cells in the bone niche. Immune evasion or suppression within the TME is one of the emerging hallmarks of cancer, which must be addressed in order to bolster antitumor immunity for optimal cancer control.¹⁹⁻²²

### Validating Syngeneic Bone Metastasis Models for Preclinical Testing of ACT.

Based on the high sensitivity and specificity of tumor-reactive T cells, T-cell therapy provides a means of eliminating normally inaccessible metastatic disease, such as CaP bone metastases. Our studies have shown that tumor-reactive T cells are capable of trafficking to metastatic bone lesions after transfer. Syngeneic bone metastasis models, e.g., B6CaP and RM1-BM, can be used to investigate trafficking, tumor infiltration, functionality, and persistence of transferred T cells in the metastatic bone niche using immunological approaches as we described previously.^{13,14,23-25} To determine whether parental RM1 or HiMyc cells share similar antigenicity with RM1-BM and B6CaP variants, respectively, we can perform in vitro cytotoxicity assays reciprocally by co-culturing RM1 (HiMyc) or RM1-BM (B6CaP) tumor-sensitized T cells with RM1-BM (B6CaP) cells or RM1 (HiMyc) cells, respectively, followed by 51Cr-release assays for their cytolytic activity. IFN-γ production and expression of Granzyme B or perforin can be assessed by intracellular staining analyses.

These studies provide baseline information on the antigenicity of these two syngeneic models of bone metastasis as well as their feasibility to be used for testing ACT and other immunotherapies (e.g., cancer vaccine). In addition, model antigen ovalbumin can be transfected into these lines, which will permit precise immune monitoring of antigen specific T cells (e.g., changes in frequency, homing, distribution, and function) derived from adoptively transferred cells or from endogenous T cell pools.

Optimized Engineering of T Cells with IL-15.

Tumor-reactive T lymphocytes (tumor infiltrating cells or draining lymph nodes) are prepared from mice with established mouse B6CaP or RM1-BM tumors and expanded as we previously described.¹⁴ Infection with lentiviruses-encoding IL-15 is performed and optimized during T cell expansion. The transduction efficiency (i.e., protein levels of the transgene in cells or media) is examined using immunoblotting or ELISA, respectively. Antibodies for CD44, CD62L and CCR7 are used together with FACS to analyze the phenotype of IL-15-engineered T cells (TCM, TEM, TE). To examine whether arming T cells with IL-15 affects their killing activity, T lymphocytes infected with Lenti-IL-15 or control virus (i.e., empty vector) are subjected to cytotoxicity assays and ELISA analysis for IFN-γ production after co-culturing with tumor targets.

MDSCs are a major element involved in the immunologic suppressive network created by interactions between the immune system and tumors.²⁶⁻³⁰ Our previous work showed that γ-chain cytokine-programmed T cells are resistant to the suppression by MDSCs. ¹⁵ To test the hypothesis that IL-15-engineered T cells display similar resistance to MDSC-mediated immunosuppression, T cells modified with or without IL-15 are stimulated in the presence of MDSCs from tumorbearing mice, followed by thymidine incorporation assays for T cell proliferation or ELISA analysis for IFN-γ production.

Homing of IL-15-Producing T cells In Vivo.

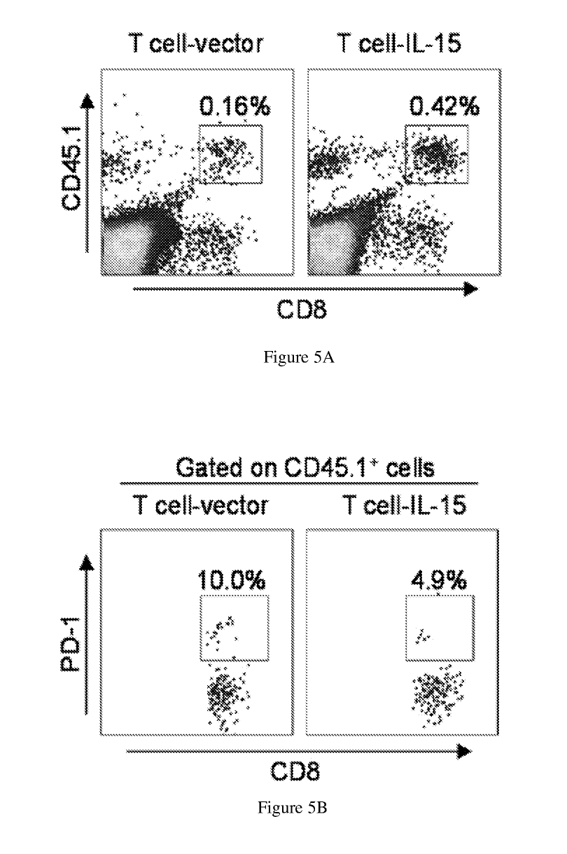

Our data show that tumor-reactive, IL-15-engineered T cells enable more efficient homing of T cells to bone metastatic niche (FIG. 5A). To determine kinetics and extent of trafficking of IL-15-producing T cells, we transferred Thy1.1+donor T cells, modified with empty vector or lenti-IL-15, to Thy1.2+recipient mice bearing bone metastases (n =3). The percentage of infused Thy1.1+ T cells in bone lesions or peripheral lymphoid tissues will be assessed at various times (days 1, 3, 7, 14, and 28) after the transfer by flow cytometric analysis. The expression levels of CCR7, CCR9, CXCR-3, CXCR-4, and CD62L that are involved in T cell trafficking, the phenotype of IL-15-engineered T cells (TCM, TEM, TE), as well as the expression of IL-15 in bone niche or tumor-infiltrating T cells will be examined. In vivo BrdU (5-Bromo-2´-Deoxyuridine) assays will assess the effect of IL-15 expression on T cell proliferation. To determine the impact of IL-15 on the long-term persistence or survival of transferred Thy1.1+ T cells in vivo, bone marrow and lymphoid tissues are harvested and analyzed two months after treatment.

Immunotherapy of Bone Metastasis of CaP with IL-15-Expressing T Cells.

Using B6CaP and RM1-BM models, we demonstrate that IL-15 engineering enhances the potency of ACT in eliminating disseminated CaP cells in the bone niche. Mice established with bone metastases (n=10) are randomized and treated with tumor-sensitized/reactive T cells that have been modified with or without IL-15. Tumor-bearing mice left untreated will serve as controls. The femurs or bone marrow cells as well as other organs (e.g., lungs, lymph nodes) will be harvested 3 weeks after tumor inoculation and examined pathologically for metastases or assessed for micrometastases using clonogenic assays. Tumor lesions or bone marrow cells will be evaluated for immune cell infiltration and cancer cell death using FACS or TUNEL assays, respectively, as we describe.²³⁻²⁵ In addition to the therapeutic setting, administration of IL-15-modified T cells to mice prior to inoculation of B6CaP or RM1-BM cells, which mimics a clinical situation in patients with a high risk of non-metastatic CaP. Generation of protective immunity demonstrates a prophylactic use of IL-15-modified T cell therapy to protect this patient population from developing bone metastases.

Depletion of host immune cells prior to T cell therapy can enhance the antitumor efficacy of transferred T cells by eliminating immune regulatory or suppressor cells (e.g., Tregs) that arrest T cells within the tumor on recognizing cognate antigen, as well as lymphocytes, that compete with the transferred cells for homeostatic cytokines.³¹⁻³³ IL-15-producing T cells are highly potent in eliminating bone metastases. Lymphodepleting regimes or preconditioning with total body irradiation (2.5 Gy) or cyclophosphamide (CYP) administration to reduce immune suppression and/or to promote homeostatic T cell expansion in vivo will be performed prior to cell transfer. Furthermore, mild irradiation treatment or CYP can induce expression of chemokine (C-X-C motif) ligand (CXCL), also known as stromal cell-derived factor (SDF)-1, in tumor lesions, which is a strong T-cell attractant³⁴ and improves homing of T cells to the tumor sites. These antitumor effects induce complete elimination of bone metastases and a tumor re-challenge can be performed to confirm the establishment of protective immune memory, which is essential for preventing relapse.

While systemic delivery of IL-15 was previously used to generate an antitumor response,^{35,36} recent reports showed that expression of IL-15 within the tumor caused rejection of established solid tumors through activated T cells³⁷ and NK cells.³⁸ A major advantage of local delivery of IL-15 to the tumor is it circumvents the cytotoxicity associated with systemic administration of IL-15. In our study the tumor specificity of T cells clearly offers a unique platform for targeted delivery of IL-15 to bone metastatic niche. In this context, IL-15 reshapes the immune environment of CaP bone metastases, which not only helps maintain the function of adoptively transferred T cells, but also mobilizes the endogenous immune elements. By employing Th1.1/Th1.2 mouse models to distinguish endogenous cells from transferred cells, we examined the immune cell infiltration (e.g., T-cells, NK-cells) of CaP bone lesion and their activation status (e.g., IFN-γ, granzyme B) using FACS and cytokine intracellular staining analysis.^{23,39} During the course of our studies we also address the question of whether exogenously administering IL-15 systemically combined with T-cell therapy is able to generate the same therapeutic efficacy as transferred T cells stably producing IL-15. It was recently reported that the therapeutic effect of IL-12 produced within the TME could not be mimicked with high doses of IL-12 delivered systemically,⁴⁰ suggesting that the kinetics of cell-delivered therapeutic agents differs from intravenous administration, consequently influencing treatment outcomes.

Evaluate the Therapeutic Efficacy of ACT Combined with Immune Checkpoint Blockade.

Immune checkpoint inhibitors, such as recently approved pembrolizumab, an antibody for programmed cell death protein (PD)-1, have shown excellent results in malignant melanoma and multiple other cancers.⁴³ The PD-1/PD-L1 pathway mediates T-cell exhaustion by antagonizing activation signaling pathways.⁴⁴⁻⁴⁷ Recent data implicate the involvement of this pathway in CaP, since PD-1 and PD-L1 have been found to be expressed in tumorinfiltrating lymphocytes obtained from prostate lesions.⁴⁸ However, administration of PD-1/PD-L1 antibodies alone do not seem to be effective in CaP patients,^{49,50} suggesting that immune checkpoint blockade may be more beneficial when combined with other immunotherapeutic regimens that can boost preexisting or generate de novo immune responses in patients. Our study showed that transferred CD8+ T cells, as expected, expressed PD-1 in bone lesions. Interestingly, IL-15 engineering significantly reduced PD-1 levels (FIG. 5B), suggesting IL-15 may render CTLs more resistant to immune suppression in the TME. Our studies show that IL-15-engineered T cells are more effective when given in combination with a PD-L1 or PD-1 inhibitor.⁵¹ The two syngeneic bone metastasis models of CaP in immune competent mice provide a unique opportunity for us to robustly evaluate the therapeutic activity of this combinatorial strategy. We injected aPD-1 (clone RMP1-14), aPD-L1 (clone 10F.9G2 from BioXcell) or rat IgG isotype control antibodies i.p. into C57BL/6 mice bearing established bone metastases on the same day of T cell therapy and continue every other day for a total of 3 doses.

Evaluate the Therapeutic Efficacy of ACT Combined with MCL-1 Inhibitor Treatment.

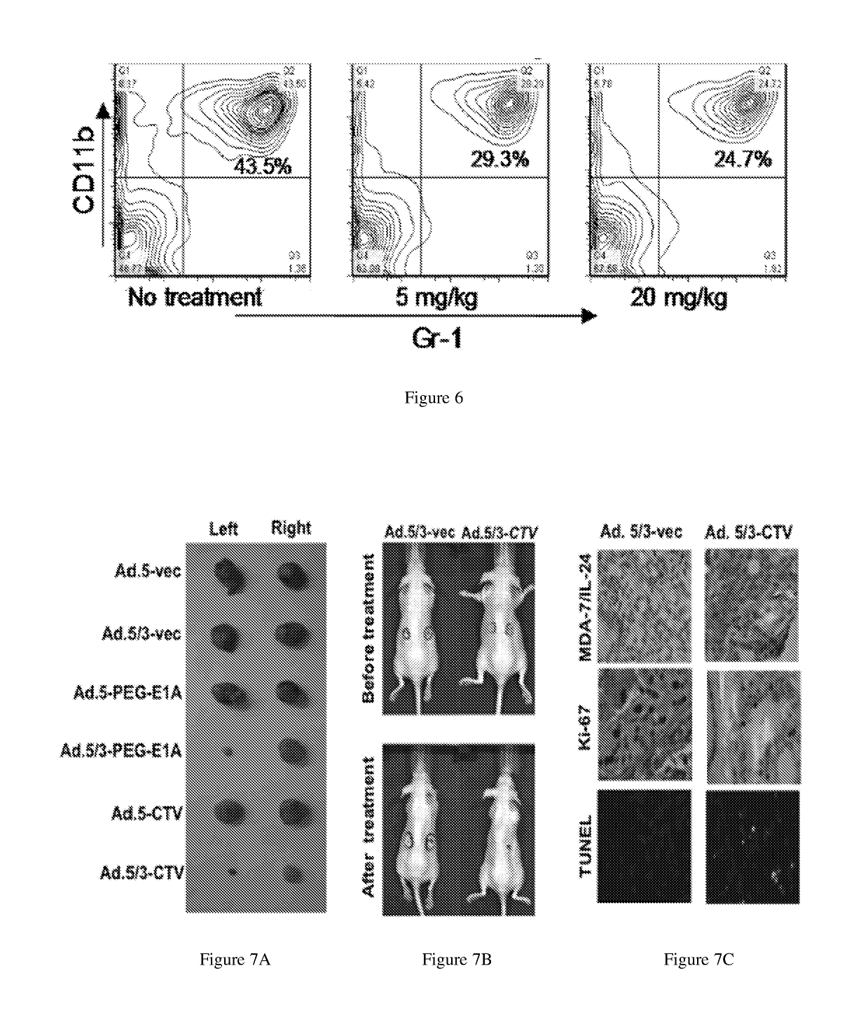

In addition to mediating tumor-associated immunosuppression, MDSCs have also been implicated in non-immunological functions, e.g., angiogenesis, tumor invasion.⁵²⁻⁵⁵ Pharmacologic targeting of MDSCs could dramatically improve immune responses in the tumor-bearing host.⁵⁶⁻⁵⁸ Interestingly, a single administration of an MCL-1 inhibitor (BI-97C1; Sabutoclax) in tumor-bearing mice substantially reduced the frequency of CD11b+Gr-1+MDSCs (FIG. 6), without inducing toxicity. Reduction of MDSCs could be explained by the direct cytotoxic effect of BI-97C1 (Sabutoclax), due to its ability to abrogate Bcl-2 protein functions. In addition to their highly potent antitumor efficacy with little cytotoxicity, and ability to sensitize CaP to other therapeutics,51,113 MCL-1 inhibitors may be used to modify the bone niche of CaP and counteract tumormediated immune suppression. Together, these results provide a solid rationale for using small molecule MCL-1 inhibitor to promote or amplify ACT-mediated antitumor toxicity by conditioning the bone metastatic environment and overcoming CaP-induced immune defects.

Using BI-97C1 (Sabutoclax) and BI-97D6, we carried out studies to define the effects of MCL-1 inhibition on the expansion and function of MDSCs in mice bearing B6CaP and RM1-BM tumors. One MCL-1 inhibitor, based on the efficacy and low toxicity, is selected to test whether inhibition of MCL-1 enhances the therapeutic efficacy of ACT, a cohort of mice established with bone metastases (n=10) is treated with T cells modified with IL-15, MCL-1 inhibitor, ACT combined with MCL-1 inhibitor, or left untreated. Administration of MCL-1 inhibitor starts before ACT treatment to sensitize CaP cells to T-cell cytotoxicity and to reduce MDSC-mediated immune suppression. Tumor response is monitored by BL1.

### Conclusion

Our data shows that IL-15-engineered T cells demonstrate significantly improved antitumor efficacy in eliminating bone metastases of CaP in immune competent syngeneic models. Support for this result is indicated by a substantial increase in the magnitude (i.e., frequency of transferred T cells in bone niche), quality (i.e., cytokine production and cytolytic activity), and duration (i.e., persistence of T cells). IL-15 engineering not only strengthens the function of transferred T cells, but also provides a direct approach via T cell delivery to reprogram the bone metastatic niche. The presence of IL-15 in the TME facilitates tumor infiltration by endogenous tumor-specific CD8₊ T cells or NK cells, augmenting their local proliferation, survival, and tumoricidal functions. The conversion of the TME into an immune-nurturing state is pivotal for enhancement of antitumor immunity. Using an MCL-1 inhibitor and/or PD-1/PD-L1 blocking antibodies to counteract the immunosuppressive pathways will improve therapeutic efficacy of T-cell therapy. It has become apparent that T cell checkpoint antagonists only overcome some of the immune-suppressive effects of the TME,¹⁵ supporting the existence of additional inhibitory mechanisms in the TME.^{50,59} The MCL-1 inhibitor and PD-1/PD-L1 antagonists target different components of the immune environment of bone metastases and thus combinatorial treatment involving the use of both agents is useful.

Previously, we have delivered therapeutic genes using viruses^{23,25,60-65} and T-cell-based therapy.^{13,14,23-25} Although there may be a concern that viral vectors induce insertional mutagenesis in hematopoietic progenitors, T cells have been shown to be far less susceptible to viral vector-induced transformation.^{66,67} While CD8+ T cells are known for their vigorous and specific responses to tumors, CD4+ T cells are also critical for long-term maintenance of antigen-activated CD8+ T cells. The relative contributions of CD8+ or CD4+ T cell subsets to tumor control can be defined by sorting cell subpopulations for testing in vivo. To broaden and peak the specific activation of transferred IL-15-engineered T cells in vivo, the simultaneous vaccination may be particularly important, given that it allows for the coordination of CD4+ T-cell help in promoting clonal expansion of tumor-specific CD8+ T cells. To achieve this, bone metastases-bearing mice are immunized with RMl cells engineered to produce a highly immunostimulatory agent that was recently developed.^{25,68,69}

### EXAMPLE 2

### Ad-Mediated MDA-7/IL-24 Expression can Eradicate Primary and Inhibit Distant CaP

MDA-7/IL-24 is established as a broad-spectrum anticancer gene capable of inducing apoptosis or toxic autophagy selectively in transformed cells of diverse origin, including CaP.⁶⁵ We evaluated Ad.5/3-CTV, a tropism modified, conditionally replication competent oncolytic adenovirus carrying mda-7/IL-24, in comparison to Ad.5-CTV in low Coxsackievirus and Adenovirus Receptor (CAR) human CaP cells, demonstrating higher efficacy in suppressing in vivo tumor growth in a nude mouse xenograft model (FIG. 7A-B) and in spontaneously developed CaP in Hi-myc transgenic mice (FIG. 7C). Ad.5/3-CTV also exerted a marked ‘bystander’ antitumor effect in vivo (FIG. 7A), thus rationalizing MDA-7/IL-24 as a therapeutic for treatment of metastatic CaP.

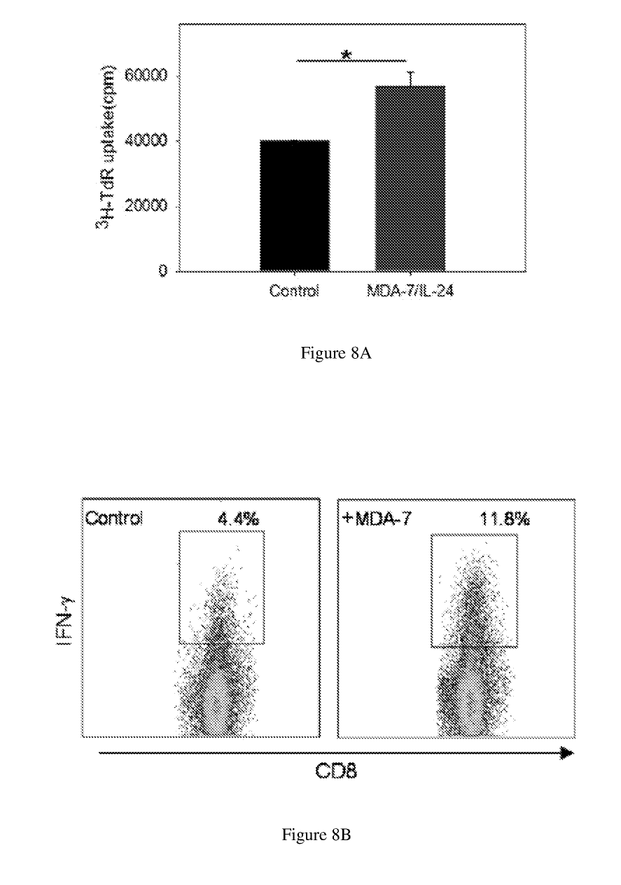

The clinical application of immunotherapy is limited by the escape mechanisms cancer cells employ to avoid immune destruction due to immunoediting (e.g., selection of non-immunogenic or antigen loss cancer cell variants) and other immunosuppressive mechanisms at the tumor site. Since cancer-specific toxicity of MDA-7/IL-24 is antigen-independent, the delivery of a cancer-suicide agent in combination with T-cells may overcome the acquired tumor resistance to T-cell therapy. Intriguingly, MDA-7/IL-24 protein enhanced the proliferation of T cells upon TCR ligation (FIG. 8A). Treatment of mice with recombinant MDA-7/IL-24 protein increased the frequency of IFN-γ-expressing CD8+ T cells (FIG. 8B), supporting a role of MDA-7/IL-24 as a pro-Th 1 cytokine.

### EXAMPLE 3

### MDA-7/IL-24-Modified T-Cell Therapy

### Characterization of the Effector Activity of MDA-7/IL-24-Modified T Cells In Vitro

We determined the effect of the IL-7/IL-15 expansion protocol on the frequency of antigen-specific T cells in vitro using RM-1-OVA tumor-sensitized T cells. Freshly isolated T cells or day 5 expanded with IL-7/IL-15 expanded day 5 T cells were stimulated with OVA peptides and subjected to ELISPOT analyses for OVA-specific IFN-γ-producing T cells. We showed that IL-7/IL-15 significantly increased the frequency of OVA-reactive T cells during cell expansion (FIG. 10A), which further supports the use of this protocol to expand tumor-reactive T cells for therapeutic applications.

To examine cytolytic activity of engineered T cells, we co-cultured RM I (antigen negative) or RMI-OVA (antigen positive) tumor cells with antigen (OVA)-specific, IL-7/IL-15 expanded/reprogrammed T cells that have been modified with either vector or MDA-7. Killing tumor cell targets by unmodified T cells or MDA-7/IL-24 expressing T cells was examined using LDH assays. We showed that MDA-7/IL-24-producing OT-I cells destroyed both RM1 and RM1-OVA tumor cells. However, unmodified OT-I cells or OT-I cells modified with an empty virus only killed RM1-OVA tumor cells (FIG. 10B). This result suggests that T cells equipped with MDA-7/IL-24 were capable of eliminating both antigen-positive as well as negative cancer cells.

We next examined tumor-sensitized T cells similarly expanded by IL-7/IL-15 and engineered by MDA-7/IL-24. As expected, MDA-7/IL-24-expressing, RM1-OVA tumor sensitized T cells displayed improved killing against RMI-OVA cells compared with unmodified or vector modified T cells (FIG. 10C). This data further supports the use of MDA-7-engineered T cells for adoptive T cell therapy to reduce cancer cell escape from antigen-dependent, immune-mediated attack.

Efficacy of MDA-7-Modified T Cell Therapy against Established Metastatic CaP

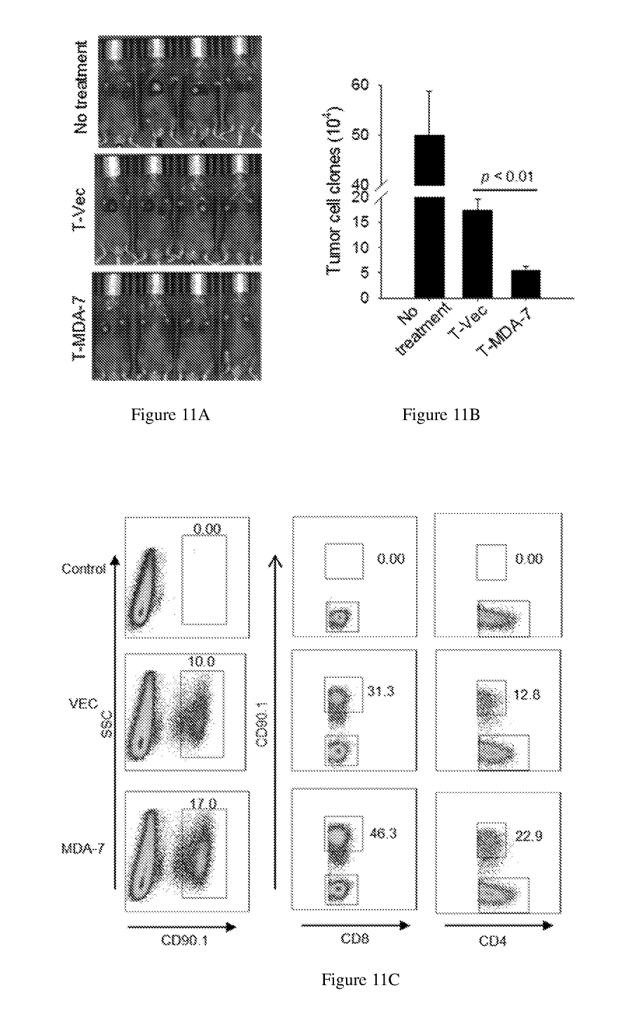

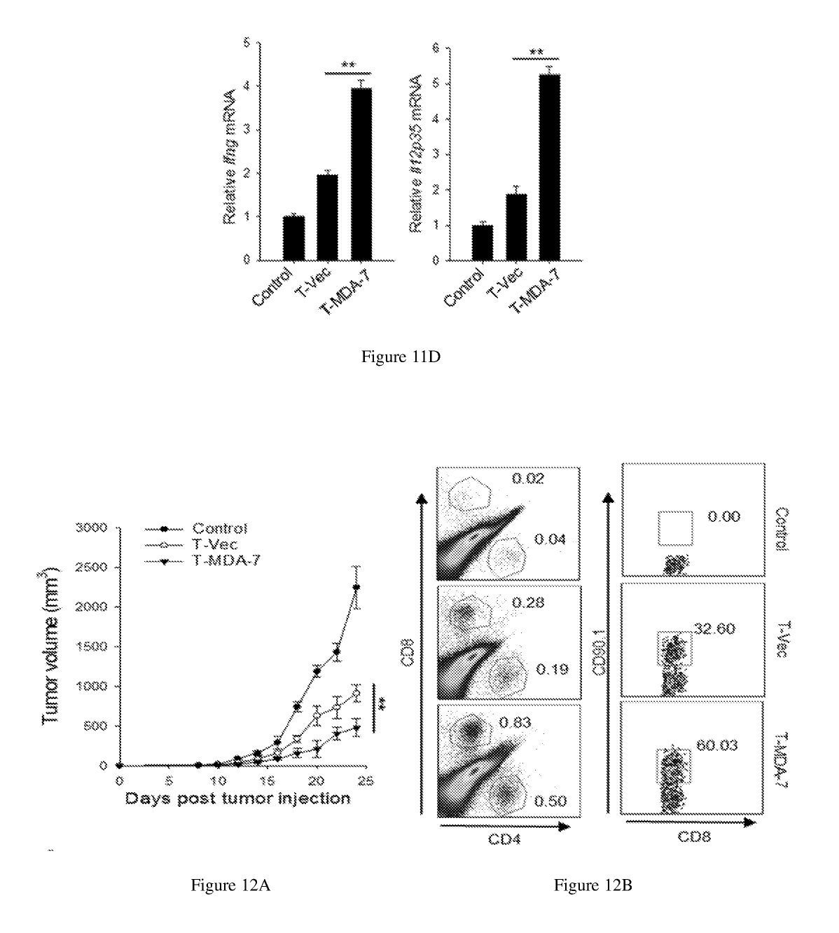

Using an experimental metastasis model of CaP that we described previously, we evaluated therapeutic activity of Cytokine Adoptive Immune Therapy (CAIT) using MDA-7/IL-24-engineered T cells. Pulmonary metastases were established in Thy1.2 (CD90.2)⁺ C57BL/6 mice by tail vein injection of RM1-luciferase (Luc) cells. These mice were then treated with IL-7/IL-15-programmed, tumor-sensitized, MDA-7-producing T cells-derived from Thy1.1 (CD90.1)⁺ mice. Bioluminescence imaging showed that MDA-7-producing T cells were more effective than those modified with vector in reducing tumor burden in the lungs (FIG. 11A). The enhanced eradication of metastases by MDA-7-modified T cell therapy was also confirmed in a clonogenic assay of lung tissues derived from treated mice (FIG. 11B).

Analyses of lung tissues with CaP metastases showed that there were significantly increased levels of CD90.1⁺ CD8⁺ or CD4⁺ T cells in mice treated with MDA-7-expressing T cells, suggesting MDA-7 modification enhanced tumor infiltration by tumor-reactive T cells (FIG. 11C). We also showed that sevel genes related to immune activation, including IFN-γ and IL-12p35 that are known to be critical for immune effector cell activation and function, were highly elevated following adoptive transfer of MDA-7-producing T cells compared to other groups (FIG. 11D), suggesting that MDA-7-modified T cell therapy may reprogram the immunosuppressive tumor environment toward the immunostimulatory one for tumor eradication.

Efficacy of MDA-7-Modified T Cell Therapy against Subcutaneous Melanoma

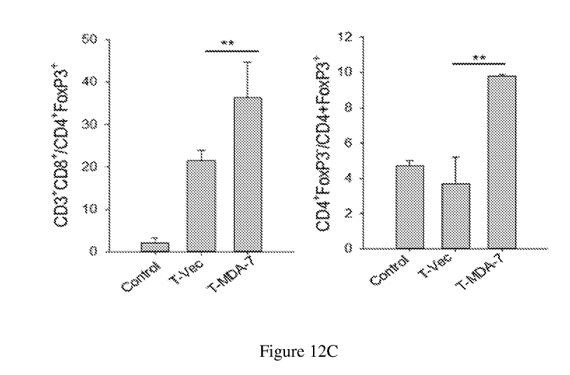

In addition to the model of lung metastases, we also evaluated the therapeutic efficacy of MDA-7 engineered T cell therapy against subcutaneously established tumors. In the first treatment setting, we initiated T cell therapy five days after inoculation of tumor cells into C57BL/6 mice. We showed that treatment with tumor-sensitized T cells that produce MDA-7 led to more effective suppression of tumor growth than vector-modified T cells (FIG. 12A). Similar therapeutic effect was also seen when treatment started nine days after tumor cell injection, even though there was a reduction of therapeutic response due to high tumor burden prior to T cell therapy (data not shown). FACS analyses of tumor tissues revealed that MDA-7 modification resulted in enhanced overall tumor infiltration by both CD4⁺ and CD8⁺ T cells (FIG. 102, left) as well as adoptively transferred CD90.1⁺ CD8⁺ T cells (FIG. 12B, right). Additionally, mice treated with MDA-7 engineered T cells showed an marked increase in the ratio of CD8⁺ T effector cells (Teff) or CD4⁺FoxP3⁻ T helper cells (Th1) vs immunosuppressive CD4⁺FoxP3⁺ regulatory T cells (Treg) in the tumor sites (FIG. 12C), which has been documented to positively correlate with therapeutic outcome in both preclinical and clinical studies.

### REFERENCES

1. Robbins, P. F., Dudley, M. E., Wunderlich, J., El-Gamil, M., Li, Y. F., Zhou, J., Huang, J., Powell, D. J., Jr. & Rosenberg, S. A. Cutting edge: persistence of transferred lymphocyte clonotypes correlates with cancer regression in patients receiving cell transfer therapy. J Immunol 173, 7125-7130 (2004).
2. Gattinoni, L., Klebanoff, C. A., Palmer, D. C., Wrzesinski, C., Kerstann, K., Yu, Z., Finkelstein, S. E., Theoret, M. R., Rosenberg, S. A. & Restifo, N. P. Acquisition of full effector function in vitro paradoxically impairs the in vivo antitumor efficacy of adoptively transferred CD8+ T cells. J Clin Invest 115, 1616-1626 (2005).
3. Berger, C., Berger, M., Hackman, R. C., Gough, M., Elliott, C., Jensen, M. C. & Riddell, S. R. Safety and immunologic effects of IL-15 administration in nonhuman primates. Blood 114, 2417-2426 (2009).
4. Huarte, E., Fisher, J., Turk, M. J., Mellinger, D., Foster, C., Wolf, B., Meehan, K. R., Fadul, C. E. & Ernstoff, M. S. Ex vivo expansion of tumor specific lymphocytes with IL-15 and IL-21 for adoptive immunotherapy in melanoma. Cancer letters 285, 80-88 (2009).
5. Sandau, M. M., Kohlmeier, J. E., Woodland, D. L. & Jameson, S. C. IL-15 regulates both quantitative and qualitative features of the memory CD8 T cell pool. J Immunol 184, 35-44 (2010).
6. Decaluwe, H., Taillardet, M., Corcuff, E., Munitic, I., Law, H. K., Rocha, B., Riviere, Y. & Di Santo, J. P. Gamma(c) deficiency precludes CD8+ T cell memory despite formation of potent T cell effectors. Proc Natl Acad Sci U S A 107, 9311-9316 (2010).
7. Manjunath, N., Shankar, P., Wan, J., Weninger, W., Crowley, M. A., Hieshima, K., Springer, T. A., Fan, X., Shen, H., Lieberman, J. & von Andrian, U. H. Effector differentiation is not prerequisite for generation of memory cytotoxic T lymphocytes. J Clin Invest 108, 871-878 (2001).
8. Wherry, E. J., Teichgraber, V., Becker, T. C., Masopust, D., Kaech, S. M., Antia, R., von Andrian, U. H. & Ahmed, R. Lineage relationship and protective immunity of memory CD8 T cell subsets. Nat Immunol 4, 225-234 (2003).
9. Kaech, S. M. & Cui, W. Transcriptional control of effector and memory CD8+ T cell differentiation. Nat Rev Immunol 12, 749-761 (2012).
10. Gattinoni, L., Klebanoff, C. A. & Restifo, N. P. Paths to stemness: building the ultimate antitumour T cell. Nat Rev Cancer 12, 671-684 (2012).
11. Klebanoff, C. A., Gattinoni, L. & Restifo, N. P. CD8+ T-cell memory in tumor immunology and immunotherapy. Immunol Rev 211, 214-224 (2006).
12. Kmieciak, M., Toor, A., Graham, L., Bear, H. D. & Manjili, M. H. Ex vivo expansion of tumor-reactive T cells by means of bryostatin 1/ionomycin and the common gamma chain cytokines formulation. Journal of visualized experiments : JoVE 47, 2381-2385 (2011).
13. Morales, J. K., Kmieciak, M., Graham, L., Feldmesser, M., Bear, H. D. & Manjili, M. H. Adoptive transfer of HER2/neu-specific T cells expanded with alternating gamma chain cytokines mediate tumor regression when combined with the depletion of myeloid-derived suppressor cells. Cancer Immunol Immunother 58, 941-953 (2009).
14. Cha, E., Graham, L., Manjili, M. H. & Bear, H. D. IL-7 +IL-15 are superior to IL-2 for the ex vivo expansion of 4T1 mammary carcinoma-specific T cells with greater efficacy against tumors in vivo. Breast cancer research and treatment 122, 359-369 (2010).
15. Payne, K. K., Toor, A. A., Wang, X. Y. & Manjili, M. H. Immunotherapy of cancer: reprogramming tumorimmune crosstalk. Clinical & developmental immunology 2012, 760965 (2012)
16. Colombetti, S., Levy, F. & Chapatte, L. IL-7 adjuvant treatment enhances long-term tumor-antigen-specific CD8+ T-cell responses after immunization with recombinant lentivector. Blood 113, 6629-6637 (2009).
17. Melchionda, F., Fry, T. J., Milliron, M. J., McKirdy, M. A., Tagaya, Y. & Mackall, C. L. Adjuvant IL-7 or IL-15 overcomes immunodominance and improves survival of the CD8+ memory cell pool. J Clin Invest 115, 1177-1187 (2005).
18. Cheever, M. A. Twelve immunotherapy drugs that could cure cancers. Immunological reviews 222, 357-368 (2008).
19. Gajewski, T. F., Meng, Y., Blank, C., Brown, I., Kacha, A., Kline, J. & Harlin, H. Immune resistance orchestrated by the tumor microenvironment. Immunol Rev 213, 131-145 (2006).
20. Rabinovich, G. A., Gabrilovich, D. & Sotomayor, E. M. Immunosuppressive strategies that are mediated by tumor cells. Annu Rev Immunol 25, 267-296 (2007).
21. Schreiber, R. D., Old, L. J. & Smyth, M. J. Cancer immunoediting: integrating immunity's roles in cancer suppression and promotion. Science 331, 1565-1570 (2011).
22. Hanahan, D. & Weinberg, R. A. Hallmarks of cancer: the next generation. Cell 144, 646-674 (2011).
23. Yi, H., Guo, C., Yu, X., Gao, P., Qian, J., Zuo, D., Manjili, M. H., Fisher, P. B., Subjeck, J. R. & Wang, X. Y. Targeting the immunoregulator SRA/CD204 potentiates specific dendritic cell vaccine-induced T cell response and antitumor immunity. Cancer Res 71, 6611-6620 (2011).
24. Qian, J., Yi, H., Guo, C., Yu, X., Zuo, D., Chen, X., Kane, J. M., 3rd, Repasky, E. A., Subjeck, J. R. & Wang, X. Y. CD204 suppresses large heat shock protein-facilitated priming of tumor antigen gp100-specific T cells and chaperone vaccine activity against mouse melanoma. J Immunol 187, 2905-2914 (2011).
25. Yu, X., Guo, C., Yi, H., Qian, J., Fisher, P. B., Subjeck, J. R. & Wang, X. Y. A multifunctional chimeric chaperone serves as a novel immune modulator inducing therapeutic antitumor immunity. Cancer Res 73, 2093-2103 (2013).
26. Rodriguez, P. C., Hernandez, C. P., Quiceno, D., Dubinett, S. M., Zabaleta, J., Ochoa, J. B., Gilbert, J. & Ochoa, A. C. Arginase I in myeloid suppressor cells is induced by COX-2 in lung carcinoma. J Exp Med 202, 931-939 (2005).
27. Pan, P.-Y., Ma, G., Weber, K. J., Ozao-Choy, J., Wang, G., Yin, B., Divino, C. M. & Chen, S.-H. Immune Stimulatory Receptor CD40 Is Required for T-Cell Suppression and T Regulatory Cell Activation Mediated by Myeloid-Derived Suppressor Cells in Cancer. Cancer Research 70, 99-108.
28. Serafini, P., Mgebroff, S., Noonan, K. & Borrello, I. Myeloid-derived suppressor cells promote crosstolerance in B-cell lymphoma by expanding regulatory T cells. Cancer Res 68, 5439-5449 (2008).
29. Srivastava, M. K., Sinha, P., Clements, V. K., Rodriguez, P. & Ostrand-Rosenberg, S. Myeloid-derived suppressor cells inhibit T-cell activation by depleting cystine and cysteine. Cancer Res 70, 68-77 (2010).
30. Hanson, E. M., Clements, V. K., Sinha, P., Ilkovitch, D. & Ostrand-Rosenberg, S. Myeloid-derived suppressor cells down-regulate L-selectin expression on CD4+ and CD8+ T cells. J Immunol 183, 937-944 (2009).
31. North, R. J. Cyclophosphamide-facilitated adoptive immunotherapy of an established tumor depends on elimination of tumor-induced suppressor T cells. J Exp Med 155, 1063-1074 (1982).
32. Gattinoni, L., Finkelstein, S. E., Klebanoff, C. A., Antony, P. A., Palmer, D. C., Spiess, P. J., Hwang, L. N., Yu, Z., Wrzesinski, C., Heimann, D. M., Surh, C. D., Rosenberg, S. A. & Restifo, N. P. Removal of homeostatic cytokine sinks by lymphodepletion enhances the efficacy of adoptively transferred tumor-specific CD8+ T cells. The Journal of experimental medicine 202, 907-912 (2005).
33. Paulos, C. M., Wrzesinski, C., Kaiser, A., Hinrichs, C. S., Chieppa, M., Cassard, L., Palmer, D. C., Boni, A., Muranski, P., Yu, Z., Gattinoni, L., Antony, P. A., Rosenberg, S. A. & Restifo, N. P. Microbial translocation augments the function of adoptively transferred self/tumor-specific CD8+ T cells via TLR4 signaling. J Clin Invest 117, 2197-2204 (2007).
34. Bleul, C. C., Fuhlbrigge, R. C., Casasnovas, J. M., Aiuti, A. & Springer, T. A. A highly efficacious lymphocyte chemoattractant, stromal cell-derived factor 1 (SDF-1). J Exp Med 184, 1101-1109 (1996).
35. Roychowdhury, S., May, K. F., Jr., Tzou, K. S., Lin, T., Bhatt, D., Freud, A. G., Guimond, M., Ferketich, A. K., Liu, Y. & Caligiuri, M. A. Failed adoptive immunotherapy with tumor-specific T cells: reversal with low-dose interleukin 15 but not low-dose interleukin 2. Cancer Res 64, 8062-8067 (2004).
36. Epardaud, M., Elpek, K. G., Rubinstein, M. P., Yonekura, A. R., Bellemare-Pelletier, A., Bronson, R., Hamerman, J. A., Goldrath, A. W. & Turley, S. J. Interleukin-15/interleukin-15R alpha complexes promote destruction of established tumors by reviving tumor-resident CD8+ T cells. Cancer Res 68, 2972-2983 (2008).
37. Liu, R. B., Engels, B., Schreiber, K., Ciszewski, C., Schietinger, A., Schreiber, H. & Jabri, B. IL-15 in tumor microenvironment causes rejection of large established tumors by T cells in a noncognate T cell receptordependent manner. Proc Natl Acad Sci USA 110, 8158-8163 (2013).
38. Liu, R. B., Engels, B., Arina, A., Schreiber, K., Hyjek, E., Schietinger, A., Binder, D. C., Butz, E., Krausz, T., Rowley, D. A., Jabri, B. & Schreiber, H. Densely granulated murine NK cells eradicate large solid tumors. Cancer Res 72, 1964-1974 (2012).
39. Guo, C., Yi, H., Yu, X., Zuo, D., Qian, J., Yang, G., Foster, B.A., Subjeck, J. R., Sun, X., Mikkelsen, R. B., Fisher, P. B. & Wang, X. Y. In situ vaccination with CD204 gene-silenced dendritic cell, not unmodified dendritic cell, enhances radiation therapy of prostate cancer. Mol Cancer Ther 11, 2331-2341 (2012).
40. Kerkar, S. P., Muranski, P., Kaiser, A., Boni, A., Sanchez-Perez, L., Yu, Z., Palmer, D. C., Reger, R. N., Borman, Z. A., Zhang, L., Morgan, R. A., Gattinoni, L., Rosenberg, S. A., Trinchieri, G. & Restifo, N. P. Tumor-specific CD8+ T cells expressing interleukin-12 eradicate established cancers in lymphodepleted hosts. Cancer Res 70, 6725-6734 (2010).
41. Uyttenhove, C., Pilotte, L., Theate, I., Stroobant, V., Colau, D., Parmentier, N., Boon, T. & Van den Eynde, B. J. Evidence for a tumoral immune resistance mechanism based on tryptophan degradation by indoleamine 2,3-dioxygenase. Nat Med 9, 1269-1274 (2003).
42. Kline, J., Brown, I. E., Zha, Y. Y., Blank, C., Strickler, J., Wouters, H., Zhang, L. & Gajewski, T. F. Homeostatic proliferation plus regulatory T-cell depletion promotes potent rejection of B16 melanoma. Clin Cancer Res 14, 3156-3167 (2008).
43. Pardoll, D. M. The blockade of immune checkpoints in cancer immunotherapy. Nat Rev Cancer 12, 252-264 (2012).
44. Parry, R. V., Chemnitz, J. M., Frauwirth, K. A., Lanfranco, A. R., Braunstein, I., Kobayashi, S. V., Linsley, P. S., Thompson, C. B. & Riley, J. L. CTLA-4 and PD-1 receptors inhibit T-cell activation by distinct mechanisms. Molecular and cellular biology 25, 9543-9553 (2005).
45. Riley, J. L. PD-1 signaling in primary T cells. Immunol Rev 229, 114-125 (2009).
46. Quigley, M., Pereyra, F., Nilsson, B., Porichis, F., Fonseca, C., Eichbaum, Q., Julg, B., Jesneck, J. L., Brosnahan, K., Imam, S., Russell, K., Toth, I., Piechocka-Trocha, A., Dolfi, D., Angelosanto, J., Crawford, A., Shin, H., Kwon, D. S., Zupkosky, J., Francisco, L., Freeman, G. J., Wherry, E. J., Kaufmann, D. E., Walker, B. D., Ebert, B. & Haining, W. N. Transcriptional analysis of HIV-specific CD8+ T cells shows that PD-1 inhibits T cell function by upregulating BATF. Nat Med 16, 1147-1151 (2010).
47. Wei, F., Zhong, S., Ma, Z., Kong, H., Medvec, A., Ahmed, R., Freeman, G. J., Krogsgaard, M. & Riley, J. L. Strength of PD-1 signaling differentially affects T-cell effector functions. Proc Nati Acad Sci USA 110, E2480-2489 (2013).
48. Tang, P. A. & Heng, D. Y. Programmed death 1 pathway inhibition in metastatic renal cell cancer and prostate cancer. Current oncology reports 15, 98-104 (2013).
49. Brahmer, J.R., Drake, C. G., Wollner, I., Powderly, J. D., Picus, J., Sharfman, W. H., Stankevich, E., Pons, A., Salay, T. M., McMiller, T. L., Gilson, M. M., Wang, C., Selby, M., Taube, J. M., Anders, R., Chen, L., Korman, A. J., Pardoll, D. M., Lowy, I. & Topalian, S. L. Phase I study of single-agent anti-programmed death-1 (MDX-1106) in refractory solid tumors: safety, clinical activity, pharmacodynamics, and immunologic correlates. J Clin Oncol 28, 3167-3175 (2010).
50. Topalian, S. L., Hodi, F. S., Brahmer, J. R., Gettinger, S. N., Smith, D. C., McDermott, D. F., Powderly, J. D., Carvajal, R. D., Sosman, J. A., Atkins, M. B., Leming, P. D., Spigel, D. R., Antonia, S. J., Horn, L., Drake, C. G., Pardoll, D. M., Chen, L., Sharfman, W. H., Anders, R. A., Taube, J. M., McMiller, T. L., Xu, H., Korman, A. J., Jure-Kunkel, M., Agrawal, S., McDonald, D., Kollia, G. D., Gupta, A., Wigginton, J. M. & Sznol, M. Safety, activity, and immune correlates of anti-PD-1 antibody in cancer. N Engl J Med 366, 2443-2454 (2012).
51. West, E. E., Jin, H. T., Rasheed, A. U., Penaloza-Macmaster, P., Ha, S. J., Tan, W. G., Youngblood, B., Freeman, G. J., Smith, K. A. & Ahmed, R. PD-L1 blockade synergizes with IL-2 therapy in reinvigorating exhausted T cells. J Clin Invest 123, 2604-2615 (2013).
52. Yang, L., Huang, J., Ren, X., Gorska, A. E., Chytil, A., Aakre, M., Carbone, D. P., Matrisian, L. M., Richmond, A., Lin, P. C. & Moses, H. L. Abrogation of TGF beta signaling in mammary carcinomas recruits Gr-1+CD11+ myeloid cells that promote metastasis. Cancer Cell 13, 23-35 (2008).
53. Yang, L., DeBusk, L. M., Fukuda, K., Fingleton, B., Green-Jarvis, B., Shyr, Y., Matrisian, L. M., Carbone, D. P. & Lin, P. C. Expansion of myeloid immune suppressor Gr+CD11b+ cells in tumor-bearing host directly promotes tumor angiogenesis. Cancer Cell 6, 409-421 (2004).
54. Shojaei, F., Wu, X., Malik, A. K., Zhong, C., Baldwin, M. E., Schanz, S., Fuh, G., Gerber, H. P. & Ferrara, N. Tumor refractoriness to anti-VEGF treatment is mediated by CD11b+Gr1+ myeloid cells. Nat Biotechnol 25, 911-920 (2007).
55. Priceman, S. J., Sung, J. L., Shaposhnik, Z., Burton, J. B., Torres-Collado, A. X., Moughon, D. L., Johnson, M., Lusis, A. J., Cohen, D. A., Iruela-Arispe, M. L. & Wu, L. Targeting distinct tumor-infiltrating myeloid cells by inhibiting CSF-1 receptor: combating tumor evasion of antiangiogenic therapy. Blood 115, 1461-1471.
56. Talmadge, J. E. Pathways mediating the expansion and immunosuppressive activity of myeloid-derived suppressor cells and their relevance to cancer therapy. Clin Cancer Res 13, 5243-5248 (2007).
57. Ko, J. S., Bukowski, R. M. & Fincke, J. H. Myeloid-derived suppressor cells: a novel therapeutic target. Curr Oncol Rep 11, 87-93 (2009).
58. Ugel, S., Delpozzo, F., Desantis, G., Papalini, F., Simonato, F., Sonda, N., Zilio, S. & Bronte, V. Therapeutic targeting of myeloid-derived suppressor cells. Curr Opin Pharmacol 9, 470-481 (2009).
59. Brahmer, J. R., Tykodi, S. S., Chow, L. Q., Hwu, W. J., Topalian, S. L., Hwu, P., Drake, C. G., Camacho, L. H., Kauh, J., Odunsi, K., Pitot, H. C., Hamid, 0., Bhatia, S., Martins, R., Eaton, K., Chen, S., Salay, T. M., Alaparthy, S., Grosso, J. F., Korman, A. J., Parker, S. M., Agrawal, S., Goldberg, S. M., Pardoll, D. M., Gupta, A. & Wigginton, J. M. Safety and activity of anti-PD-L1 antibody in patients with advanced cancer. N Engl J Med 366, 2455-2465 (2012).
60. Gao, P., Sun, X., Chen, X., Wang, Y., Foster, B. A., Subjeck, J., Fisher, P. B. & Wang, X. Y. Secretable chaperone Grp170 enhances therapeutic activity of a novel tumor suppressor, mda-7/IL-24. Cancer Res 68, 3890-3898 (2008).
61. Dent, P., Yacoub, A., Hamed, H. A., Park, M. A., Dash, R., Bhutia, S. K., Sarkar, D., Wang, X. Y., Gupta, P., Emdad, L., Lebedeva, I. V., Sauane, M., Su, Z. Z., Rahmani, M., Broaddus, W. C., Young, H. F., Lesniak, M. S., Grant, S., Curiel, D. T. & Fisher, P. B. The development of MDA-7/IL-24 as a cancer therapeutic. Pharmacology & therapeutics 128, 375-384 (2010).
62. Dash, R., Azab, B., Shen, X. N., Sokhi, U. K., Sarkar, S., Su, Z. Z., Wang, X. Y., Claudio, P. P., Dent, P., Dmitriev, I. P., Curiel, D. T., Grant, S., Sarkar, D. & Fisher, P. B. Developing an effective gene therapy for prostate cancer: New technologies with potential to translate from the laboratory into the clinic. Discovery medicine 11, 46-56 (2011).
63. Zuo, D., Yu, X., Guo, C., Yi, H., Chen, X., Conrad, D. H., Guo, T. L., Chen, Z., Fisher, P. B., Subjeck, J. R. & Wang, X. Y. Molecular chaperoning by glucose-regulated protein 170 in the extracellular milieu promotes macrophage-mediated pathogen sensing and innate immunity. Faseb J 26, 1493-1505 (2012).
64. Bhutia, S. K., Mukhopadhyay, S., Sinha, N., Das, D. N., Panda, P. K., Patra, S. K., Maiti, T. K., Mandal, M., Dent, P., Wang, X. Y., Das, S. K., Sarkar, D. & Fisher, P. B. Autophagy: cancer's friend or foe? Adv Cancer Res 118, 61-95 (2013).
65. Zuo, D., Yu, X., Guo, C., Wang, H., Qian, J., Yi, H., Lu, X., Lv, Z. P., Subjeck, J. R., Zhou, H., Sanyal, A. J., Chen, Z. & Wang, X. Y. Scavenger receptor a restrains T-cell activation and protects against concanavalin A-induced hepatic injury. Hepatology 57, 228-238 (2013).
66. Recchia, A., Bonini, C., Magnani, Z., Urbinati, F., Sartori, D., Muraro, S., Tagliafico, E., Bondanza, A., Stanghellini, M. T., Bernardi, M., Pescarollo, A., Ciceri, F., Bordignon, C. & Mavilio, F. Retroviral vector integration deregulates gene expression but has no consequence on the biology and function of transplanted T cells. Proc Natl Acad Sci U S A 103, 1457-1462 (2006).
67. Newrzela, S., Cornils, K., Li, Z., Baum, C., Brugman, M. H., Hartmann, M., Meyer, J., Hartmann, S., Hansmann, M. L., Fehse, B. & von Laer, D. Resistance of mature T cells to oncogene transformation. Blood 112, 2278-2286 (2008).
68. Yu, X. & Wang, X. Y. Engineering Grp170-based immune modulators for cancer immunotherapy. Oncoimmunology 2, e24385 (2013).
69. Yu, X., Subjeck, J. R. & Wang, X. Y. Integrating a ‘danger’ signal into molecular chaperoning to improve vaccination against cancer. Expert Rev Vaccines 12, 581-583 (2013).

While the invention has been described in its preferred embodiments, those of skill in the art will recognize the invention can be practiced with variations within the spirit and scope of the appended claims.

1. A T lymphocyte genetically modified to express Melanoma differentiation associated gene-7/Interleukin-24 (MDA-7/IL-24) or a functional-conservative derivative thereof.

2. The T lymphocyte of claim 1, wherein said MDA-7/IL-24 or functional-conservative derivative thereof comprises a FMS-like tyrosine kinase 3 (Flt-3) secretory motif.

3. The T lymphocyte of claim 1, wherein said T lymphocyte is genetically modified to further express at least one of IL-15, IL-12, and MDA-5.

4. A composition for adoptive cell transfer comprising T lymphocytes according to claim 1 and a pharmaceutically acceptable carrier.

5. The composition of claim 4 further comprising one or more chemotherapeutic or radiotherapeutic agents.

6. A method of treating and preventing recurrence of cancer and/or cancer metastasis in a subject in need thereof, comprising
administering to the subject a therapeutically effective amount of a composition for adoptive cell transfer comprising T lymphocytes that express MDA-7/IL-24 or a functional-conservative derivative thereof.

7. The method of claim 6, wherein said T lymphocytes are isolated from said subject and genetically modified to express MDA-7/IL-24 or a functional-conservative derivative thereof.

8. The method of claim 6, wherein said cancer is selected from the group consisting of prostate, brain, breast, pancreatic, liver, kidney, lung, spleen, gall bladder, anal, testicular, ovarian, cervical, skin, bone, blood, and colon cancer.

9. The method of claim 6, wherein said metastatic cancer has spread to at least one bone of said subject.

10. The method of claim 6, wherein said MDA-7/IL-24 or functional-conservative derivative thereof comprises a FMS-like tyrosine kinase 3 (Flt-3) secretory motif.

11. The method of claim 6, wherein said T lymphocyte is genetically modified to further express at least one of IL-15, IL-12, and MDA-5.

12. The method of claim 6, further comprising a step of administering T lymphocytes, different from said T lymphocytes that express MDA-7/IL-24 or a functional-conservative derivative thereof, genetically modified to express at least one of IL-15, IL-12, and MDA-5.

13. The method of claim 6, further comprising a step of administering one or more of a chemotherapeutic or radiotherapeutic agent.

14. The method of claim 6, further comprising a step of administering at least one of an immune checkpoint inhibitor and a Mcl-1 inhibitor.

15. A T lymphocyte genetically modified to express IL-15 or a functional-conservative derivative thereof.

16. The T lymphocyte of claim 15, wherein said T lymphocyte is genetically modified to further express at least one of MDA-7/IL-24, IL-12, and MDA-5.

17. A composition for adoptive cell transfer comprising T lymphocytes according to claim 15 and a pharmaceutically acceptable carrier.

18. The composition of claim 17 further comprising one or more chemotherapeutic or radiotherapeutic agents.

19. A method of treating and preventing recurrence of cancer and/or cancer metastasis in a subject in need thereof, comprising
administering to the subject a therapeutically effective amount of a composition for adoptive cell transfer comprising T lymphocytes that express IL-15 or a functional-conservative derivative thereof.

20. The method of claim 19, wherein said T lymphocytes are isolated from said subject and genetically modified to express IL-15 or a functional-conservative derivative thereof.

21. The method of claim 19, wherein said cancer is selected from the group consisting of prostate, brain, breast, pancreatic, liver, kidney, lung, spleen, gall bladder, anal, testicular, ovarian, cervical, skin, bone, blood, and colon cancer.

22. The method of claim 19, wherein said metastatic cancer has spread to at least one bone of said subject.

23. The method of claim 19, wherein said T lymphocyte is genetically modified to further express at least one of MDA-7/IL-24, IL-12, and MDA-5.

24. The method of claim 19, further comprising a step of administering T lymphocytes, different from said T lymphocytes that express MDA-7/IL-24 or a functional-conservative derivative thereof, genetically modified to express at least one of IL-15, IL-12, and MDA-5.

25. The method of claim 19, further comprising a step of administering one or more of a chemotherapeutic or radiotherapeutic agent.

26. The method of claim 20, further comprising a step of administering at least one of an immune checkpoint inhibitor and a Mcl-1 inhibitor.


SEQUENCE LISTING (4 sequences)

SEQ ID NO: 1 - DNA, 621 bp, Homo sapiens
atgaattttcaacagaggctgcaaagcctgtggactttagccagacccttctgccctcctttgctggcgacagcctctcaaatgcagatggttgtgctcccttgcctgggttttaccctgcttctctggagccaggtatcaggggcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctctga

SEQ ID NO: 2 - protein, 206 aa, Homo sapiens
MNFQQRLQSLWTLARPFCPPLLATASQMQMVVLPCLGFTLLLWSQVSGAQGQEFHFGPCQVKGVVPQKLWEAFWAVKDTMQAQDNITSARLLQQEVLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 3 - DNA, 312 bp, Homo sapiens
gagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctctga

SEQ ID NO: 4 - protein, 103 aa, Homo sapiens
ESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL